(19) United States
(12) Patent Application Publication
(10) Pub. No.: US 2025/0152332 A1
(43) Pub. Date: May 15, 2025

(54) SURGICAL SYSTEM AND METHODS OF USE

(71) Applicant: Medtronic, Inc., Minneapolis, MN (US)

(72) Inventors: Jonathan T. Goodman, Robbinsdale, MN (US); Shanthi John Lawrence, Mounds View, MN (US); Charlene X. Yuan, Plymouth, MN (US); Daniel R. Lexcen, Blaine, MN (US)

(21) Appl. No.: 18/945,912

(22) Filed: Nov. 13, 2024

Related U.S. Application Data
(60) Provisional application No. 63/598,246, filed on Nov. 13, 2023.

(51) Int. Cl. A61F 2/12; A61F 2/00

(52) U.S. Cl. CPC A61F 2/12; A61F 2/0077; A61F 2210/0004; A61F 2250/0036; A61F 2250/0067

(57) ABSTRACT

An implantable medical device includes a mesh substrate. A first coating that covers at least a portion of the substrate. The first coating includes a first biodegradable or bioresorbable polymer. The first coating has a chemotherapeutic agent dispersed in the first polymer such that the first polymer releases the agent as the first polymer degrades. A second coating covers at least a portion of the first coating. The second coating includes a second biodegradable or bioresorbable polymer. The second coating has an antibiotic agent dispersed in the second polymer such that the second polymer releases the agent as the second polymer degrades. The first coating is configured to release the chemotherapeutic agent over about 10-180 days and the second coating is configured to release the antibiotic agent over about 3-10 days.

### RELATED APPLICATIONS

This application claims the benefit of U.S. Provisional Application Ser. No. 63/598,246, filed Nov. 13, 2023, the entire contents of each of which are incorporated herein by reference.

### TECHNICAL FIELD

The present disclosure generally relates to an implantable medical device, wherein the implantable medical device includes one or more biodegradable or bioresorbable layers that are configured to release antibiotic and chemotherapeutic agents as the one or more layers degrade.

### BACKGROUND

Cancerous tissues within the body may be treated in a variety of different ways including through surgery, chemotherapy, radiation, hormonal, targeted, immunotherapy, transplant, and the like. Chemotherapy involves the use of anti-cancer drugs (chemotherapy agents) to destroy cancer cells. Chemotherapy agents can be used to treat multiple use cases of cancer, including, for example, breast cancer, skin cancer, colorectal/bowel cancer and lung cancer. Surgery is another common approach where the cancerous tissue or tumor is surgically removed from the patient's body. In some examples, chemotherapy is administered after surgical intervention. For example, with breast cancer, reconstruction of breast tissue is often considered in combination with chemotherapy.

Chemotherapeutic agents like Paclitaxel (PTX), doxorubicin (DOX) and cisplatin, which are widely used for chemotherapy of various solid tumors, have been reported to have excellent antitumor effects. These drugs or other appropriate drug candidates can be used for combinatorial or synergetic action. Paclitaxel has undergone extensive clinical development and has been approved as an effective antitumor drug. Doxorubicin has established enhanced cell kill with tumor-selective activation and hence tumor-selective cell kill. It has decreased cardiotoxicity and decreased resistance to prolonged treatment. Moreover, it has been found that there is no drug interaction reported between cisplatin and certain antibiotics. One challenge with drug administration is the delivery technique and obtaining appropriate efficacy.

In 2016, the American Society of Plastic Surgeons has reported that more than 109,256 breast reconstruction procedures were performed in the United States alone. In a study including 3,128 breast cancer patients a cumulative risk of recurrence of 21% after 15 years of follow-up was published, in another report of 36,924 women with breast cancer, an incidence rate of 15.53 per 1000 person-years, has been documented. This data demonstrates the propensity of the disease and the need to address this patient population with a heretofore unknown combinatorial chemotherapeutic solution.

### SUMMARY

In one embodiment, in accordance with the principles of the present disclosure, an implantable medical device (IMD) that includes a substrate that includes a biodegradable or bioresorbable material. The IMD includes a first layer on at least a portion of the substrate, the first layer having a first biodegradable or bioresorbable polymer and a chemotherapeutic agent dispersed in the first polymer, where the first layer is configured to release the chemotherapeutic agent over a first period of time of at least 10 days as the first polymer degrades. The IMD also includes a second layer on at least a portion of the first coating or the substrate. The second layer having a second biodegradable or bioresorbable polymer and at least one antibiotic agent dispersed in the second polymer, where the second layer is configured to release the at least one antibiotic agent over a second period of time of at least 1 day as the second polymer degrades.

In another embodiment, in accordance with the principles of the present disclosure, a surgical system that includes a surgical implant (e.g., breast implant), and a IMD in form of a sheet or pouch configured to at least partially surround the surgical implant. The IMD includes a substrate that includes a biodegradable or bioresorbable material and a first layer on at least a portion of the substrate. The first layer having a first biodegradable or bioresorbable polymer and a chemotherapeutic agent dispersed in the first polymer, where the first layer is configured to release the chemotherapeutic agent over a first period of time of at least 10 days as the first polymer degrades. The IMD also includes a second layer on at least a portion of the first coating or the substrate. The second layer having a second biodegradable or bioresorbable polymer and at least one antibiotic agent dispersed in the second polymer, where the second layer is configured to release the at least one antibiotic agent over a second period of time of at least 1 day as the second polymer degrades.

In another embodiment, in accordance with the principles of the present disclosure, an IMD having a bioresorbable mesh substrate and a first coating that covers at least a portion of the substrate. The first coating having a first biodegradable or bioresorbable polymer and a chemotherapeutic agent dispersed in the first polymer such that the first polymer releases the chemotherapeutic agent as the first polymer degrades, where the first coating is configured to release the chemotherapeutic agent over a period of more than 10 days. The IMD also includes a second coating that covers at least a portion of the first coating. The second coating having a second biodegradable or bioresorbable polymer, the second coating having at least one antibiotic agent dispersed in the second polymer such that the second polymer releases the at least one antibiotic agent as the second polymer degrades, where the second coating is configured to release the second active agent in over at least about 3 days. The IMD further including a third coating that covers at least a portion of the second coating. The third coating having a third biodegradable or bioresorbable polymer, the third coating having a hemostatic agent dispersed in the third polymer such that the third polymer releases the hemostatic agent as the third polymer degrades, where the third coating is configured to release the hemostatic agent in less than 3 days.

Additional features and advantages of various embodiments will be set forth in part in the description that follows, and in part will be apparent from the description, or may be learned by practice of various embodiments, such as, for example, substantiating polymer mesh conjugated with stem cell seeding, heat shock proteins and growth factors. In some embodiments, an implant is provided wherein the implant's size, volume and shape profile will be designed to meet the clinical needs with three different sizes-small, medium, and large pouches/pockets, sheets and patches. The objectives and other advantages of various embodiments will be realized and attained by means of the elements and combinations particularly pointed out in the description and appended claims.

### BRIEF DESCRIPTION OF THE DRAWINGS

The present disclosure will become more readily apparent from the specific description accompanied by the following drawings, in which:

FIG. 1 is a plan view of components of a surgical system, in accordance with the principles of the present disclosure;

FIG. 2 is an example perspective view of the implantable medical device that may be used with the system of FIG. 1;

FIG. 3 is a side view of the is an example perspective view of the implantable medical device of that may be used with the system FIG. 1;

FIG. 4 is a cross-sectional view of an example implantable medical device that may be used in the system of FIG. 1;

FIGS. 5-7 are close up views of representative components shown in FIG. 4;

FIGS. 8-14 are cross-sectional views of additional example implantable medical devices that may be used in the system of FIG. 1;

FIG. 15 is a chart showing tests results from testing of one embodiment of the component of the surgical system shown in FIG. 1, in accordance with the principles of the present disclosure; and

FIG. 16 is a chart showing tests results from testing of the component discussed in connection with FIG. 15.

### DETAILED DESCRIPTION

For the purposes of this specification and appended claims, unless otherwise indicated, all numbers expressing quantities of ingredients, percentages or proportions of materials, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims. In some embodiments, the fabrication process of the chemotherapeutic agent(s) and/or the active agent(s) discussed herein can be tailored for thermal activation or other process.

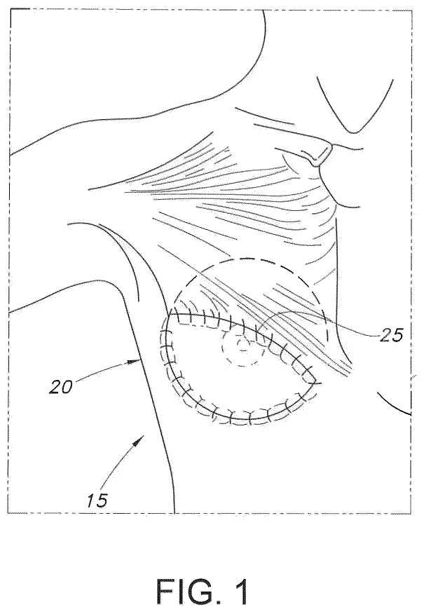

FIGS. 1-3 illustrate various parts and features of a surgical system 15 that includes an implantable medical device (IMD) 20 that is at least partially or fully biodegradable or bioresorbable. As used herein, the term “biodegradable” refers to, for example, a material that can be broken down or degraded by a bodily fluid and discarded as waste from the body and/or a material that can be broken down or degraded by a living organism. Thus, “non-biodegradable” can refer to a material that cannot be broken down or degraded by a bodily fluid and/or cannot be broken down or degraded by a living organism. As used herein the term “resorbable” refers to, for example, a material that can be broken down or degraded by a bodily fluid and assimilated within the body. Thus, a “non-resorbable” material as used herein can refer to, for example, a material that cannot be broken down or degraded by bodily fluid and assimilated within the body.

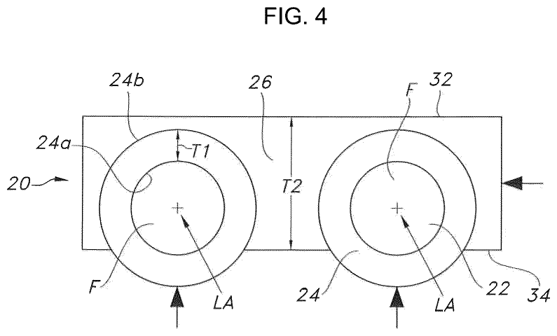

The IMD 20 may be used independent of another device or may be used in conjunction with another implantable medical device or surgical implant (e.g., device 25) such as a non-biodegradable breast implant as shown in FIGS. 1-3. IMD 20 is a multi-layered device that includes substrate 22 (FIG. 4) such as a mesh, film, or sheet along with multiple discrete biodegradable or bioresorbable layers (e.g., coatings) thereon. Each layer includes at least one therapeutic agent dispersed in a biodegradable or bioresorbable polymer carrier configured to carry and release the various agents at different release rates to enhance surgical outcomes, including, for example, long-term (e.g., weeks or months) and short-term (e.g., days) delivery of the different therapeutic agents. More specifically, IMD 20 may include at least one layer that includes one or more chemotherapeutic agents in a biodegradable or bioresorbable polymer carrier and at least one layer that includes one or more antibiotics dispersed in a different biodegradable or bioresorbable polymer carrier such that the chemotherapeutic agent and the antibiotic are released at different rates to both improve the short-term health at the surgical site and provide long-term chemotherapy treatment over an extended period of time.

IMD 20 may be particularly advantageous in the treatment of surgical procedures where cancer tissue is removed from the body. One such procedure may include breast reconstruction following a mastectomy, in which implantable medical device 25 is a breast implant and IMD 20 is configured to cover or engage at least a portion of device 25. For example, IMD 20 can be used to anchor device 25 wherein device 25 is a breast implant to tissue in an area of a patient that has had a cancerous tumor removed and/or is in the need of cancerous treatment. As such, IMD 20 may be used to provide and promote short-term wound healing via antibiotics in conjunction with site specific long-term chemotherapy to help inhibit the regrowth of cancer tissue. Localized chemotherapy via local controlled release from IMD 20 may lead to amplified therapeutic efficacies, as well as reduced side effects of systemic administration. Tumors close to blood vessels, vital organs or unreachable contours can be treated, customizing to prepectoral, subpectoral breast fascia or muscular regions matching the tissue profile, in order to prevent foreign body response and accelerate therapeutic outcomes with localized delivery.

In some examples, IMD 20 may be configured to be coupled, applied, partially encase and/or surround a non-biodegradable or non-bioresorbable implantable medical device 25. Device 25 may include, for example, a breast implant, as shown in FIG. 1, a reconstructive implant, a surgical implant or marker such as a biopsy marker, or other surgically implanted device such as a cardiac monitor, pacemaker, or the like that is intended to be implanted and left in the body or until it is removed. In some such examples, IMD 20 may be discrete and separable (e.g., prior to implant) from medical device 25 and used to improve the surgical outcome of implanting device 25. For example, in embodiments wherein device 25 is a breast implant, IMD 20 may be configured to support device 25 upon implantation of device 25 within a patient to release antibiotics and chemotherapeutic agents in an area surrounding device 25, while simultaneously providing support for device 25 within the patient. That is, IMD 20 is configured to connect with tissue adjacent to device 25 to prevent or reduce relative movement between device 25 and the tissue, while at the same time releasing antibiotics and chemotherapeutic agents in the area surrounding device 25 at an intended rate. System 15 thus provides both structural support and medicinal therapy for patients that receive implantable medical device 25.

IMD 20 can have a variety of different configurations, such as, for example, a variety of different shapes and/or sizes. For example, whether IMD 20 is used independently (without device 25) or in combination with device 25, IMD 20 can be provided with a size and/or shape to fit the desired treatment site. In addition to size and shape configurations, whether IMD 20 is used independently (without device 25) or in combination with device 25, IMD 20 may also be configured such that a coating or layer of IMD 20 that includes antibiotic or bleed control agents will directly contact or engage tissue of a patient that for more immediate release of such agents along with a layer or coating containing a chemotherapeutic agent (e.g., below the layer containing the antibiotic or bleed control agent) to provide more extended release of the chemotherapeutic agent to a target tissue such as tissue that had a tumor removed and/or is in need of cancerous treatment. Additionally, or alternatively, IMD 20 can be provided with a size and/or shape or other configuration that can provide the functionality of supporting and immobilizing medical device 25 at a treatment site within a patient's body. In some embodiments, IMD 20 can be configured to partially or fully surround and/or enclose at least a portion of device 25 to hold and/or support device to prevent relative movement between device 25 and tissue. In other examples, IMD 20 can be used independently, without device 25 as a standalone system and still obtain several of the advantages described. That is, in some examples, system 15 includes IMD 20, but does not include device 25.

In some embodiments wherein device 25 is removable, IMD 20 is sized and/or shaped to improve the removability of device 25 after the treatment has been completed. It would be appreciated by one of ordinary skill in the art that the size and/or shape of IMD 20 can be selected to conform to the size and/or shape of device 25. In embodiments wherein device 25 is a breast implant, IMD 20 may be sized and/or shaped to support device 25 by covering all or a portion of device 25. However, when IMD 20 is used independently (without device 25), the size and/or shape of IMD 20 can be customized without considerations to device 25 and instead to consideration, such as, for example, to the size and shape of tissue/anatomy of a patient that has had a tumor removed and/or is in need of cancerous treatment.

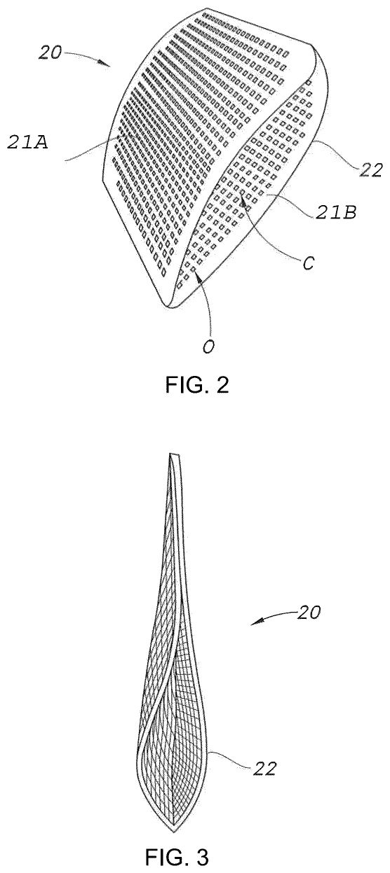

IMD 20 may be provided in the form of a sheet (e.g., knitted mesh). The sheet may be bendable and/or pliable to conform the shape of the sheet to the shape of device 25, as shown in FIG. 1, or to the shape of the implant site. In some embodiments, IMD 20 includes only one sheet, such as, for example, only first piece 21A of FIG. 2, and in other embodiments, IMD 20 includes at least two sheets, such as, for example, second piece 21B, in addition to first piece 21A, as shown in FIGS. 2 and 3. For example, in some embodiments IMD 20 may include first piece 21A and second piece 21B such that first and second pieces 21A, 21B form a pocket or pouch. In some embodiments, first and second pieces 21A, 21B are joined along three sides of the pocket or envelope to form a cavity C (e.g., FIG. 2) configured to at least partially receive and surround medical device 25 (e.g., breast implant) as discussed herein. In some embodiments, second piece 21B is separate from first piece 21A and/or is fixably or removably attached to first piece 21A through appropriate means (e.g., sewn, heat welded, ultrasonically welded, bonded, knitted, glued or the like). In some embodiments, at least one of pieces 21A, 21B includes tabs for suturing device 25 in place.

In some embodiments, first piece 21A is identical to second piece 21B, with each piece including a substrate and therapeutic coatings described herein. In other embodiments, first piece 21A may be different than second piece 21B and/or first piece 21A and second piece 21B may be constructed with different layer constructions. For example, first piece 21A may include a substrate and coating containing a chemotherapeutic agent and second piece 21B may include a substrate and a coating containing an antibiotic agent and the two pieces joined together to produce IMD 20. As discussed further below with reference to FIG. 4, the respective surfaces of IMD 20 may be constructed based on whether the surface is intended to be oriented toward device 25 or toward surrounding tissue of the patient. The surface or layer configured to face the surrounding tissue may degrade earlier or preferentially compared to the surface or layer facing medical device 25.

The components of IMD 20 can be fabricated from biologically acceptable materials suitable for medical applications, including metals, synthetic polymers, allografts, xenografts, isografts, ceramics and bone material and/or their composites, depending on the particular application and/or preference of a medical practitioner. Temporary tissue expanders and artificial shells before silicone exchange can also be fabricated and included with the layered coating polymer materials discussed herein. Various components of IMD 20 may have material composites, including the above materials, to achieve various desired characteristics such as release characteristics, strength, rigidity, elasticity, compliance, biomechanical performance, durability and radiolucency or imaging preference. The components of IMD 20, individually or collectively, may also be fabricated from a heterogeneous material such as a combination of two or more of the below-described materials. The components of IMD 20 may be monolithically formed, integrally connected or include fastening elements and/or instruments, as described herein. The described coatings may be distinct layers or laminate coatings that are fixably adhered or readily separable from one another.

FIGS. 4-7 show various examples of IMD 20 and the components or features thereof in more detail. While the layers one the substrate are generally described as coatings that are adhered to the substrate or adjacent layers throughout this disclosure, in some embodiments one or more of the layers may represent discrete structures that are assembled (e.g., stacked, folded, stitched, laminated, or heat welded) together to form the IMD 20. Accordingly, constructions where IMD 20 includes multiple discrete layers that are not directly bonded together are also envisioned within this disclosure.

FIG. 4 is a cross-sectional view of one example IMD 20 that includes a substrate 22 and a first coating 24 that includes a first biodegradable or bioresorbable polymer and a first therapeutic agent such as one or more chemotherapeutic agents dispersed in the first polymer and a second coating 26 that includes a second biodegradable or bioresorbable polymer and a second therapeutic agents such as one or more antibiotics dispersed in the second polymer. As used herein, the designation of a “first” or “second” coating is used for purposes of differentiation and not intended, unless specified, to imply the order of application or placement of the coatings or layers. Hence while second coating 26 is depicted in FIG. 4 as being on first coating 24, in other examples, first coating 24 may be positioned on second coating 26 such that second coating 26 is positioned between substrate 22 and first coating 24. Further, description of a coating being “on” or “covering” another component may describe the coating being directly on (i.e., with no intermediate coatings or layers therebetween) or indirectly on (e.g., with one or more intermediate coatings or layers therebetween) the component. Hence, in FIG. 4 second coating 26 can be characterized as being on substrate 22 or on first coating 24.

Substrate 22 may act as an anchoring system configured help secure IMD 20, medical device 25 (if present), or both to the surrounding tissue. Substrate 22 may also act as a foundation or scaffolding upon which the described coatings may be applied that at least partially coat substrate 22. Substrate 22 may be constructed of materials configured to help promote wound health and healing compared to implant procedures wherein device 25 is otherwise implanted without the use of IMD 20.

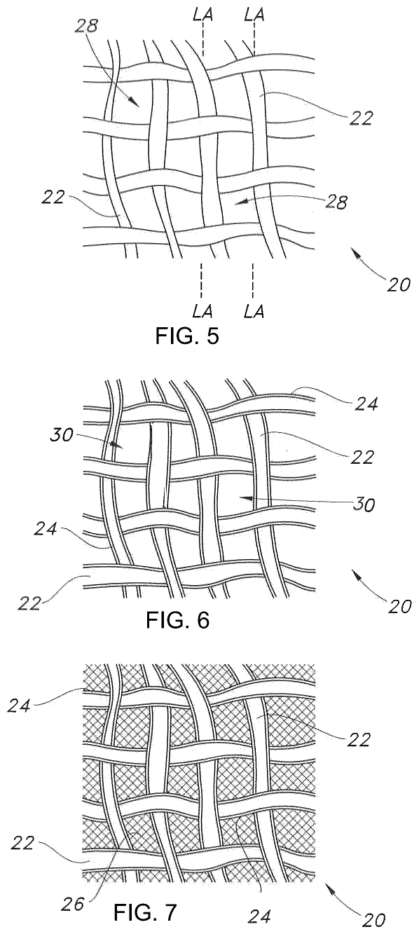

Substrate 22 may be provided in the form of a mesh, film, or sheet, preferably a biodegradable or bioresorbable mesh that can be implanted and absorbed by the body over an extended period of time (e.g., several months). In some embodiments, substrate 22 does not begin to degrade until coatings 24, 26 sufficiently degrade to expose substrate 22 to fluid and surrounding tissue. The substrate 22 may be constructed to include one or more layers (e.g., mesh layers) and/or one or more sheets that can be folded, stacked, or at least partially secured together (e.g., FIG. 2). Substrate 22 may be porous or otherwise include one or more apertures configured to promote tissue ingrowth or fluid transmission through substrate 22. For example, substrate 22 may be in the form of a mesh is a web or fabric (e.g., FIG. 5) with a construction of knitted, braided, woven or non-woven filaments or fibers F that are interlocked in such a way to create a fabric or a fabric-like material that includes a matrix of filaments that define apertures 28 between adjacent fibers F. As described further below, first and second coatings 24 and 26 may partially or fully occluded apertures 28 in the final construction of IMD 20.

In some embodiments, substrate 22 may be composed of biocompatible, biodegradable or bioresorbable materials. Constructing substrate 22 out of bioresorbable materials allows IMD 20 to be implanted and slowly absorbed by the body over a set extended period of time (e.g., several months) to deliver the one or more therapeutic agents described herein over the entirety, or a portion of, the extended period of time. This allows for rapid or prolonged release of the various therapeutic agents as desired without the need to subsequently remove any materials from the body. Suitable biodegradable or bioresorbable materials for substrate 22 may include, but are not limited to, polymeric and/or non-polymeric materials, such as, for example, one or more poly(alpha-hydroxy acids), poly(lactide-co-glycolide) (PLGA), polylactide (PLA), poly(L-lactide), polyglycolide (PG), polyethylene glycol (PEG) conjugates of poly(alpha-hydroxy acids), polyorthoesters (POE), polyaspirins, polyphosphazenes, decellularized extracellular matrix (dECM), collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, POE, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteramides, tyrosine polyarylates, polyetheretherketone, polymethylmethacrylate, silicone, hyaluronic acid, chitosan, or combinations thereof. In one embodiment, substrate 22 comprises Glycoprene, which is sold by Poly-Med, Inc. As used herein, the term “glycoprene” or “Glycoprene” refers to Glycoprene®) or Glycoprene II®. Glycoprene® can refer to different variations of the material sold under the trade name Glycoprene®, such as, for example, Glycoprene® 6829, Glycoprene® 8609 and Glycoprene® 7027.

In some embodiments, IMD 20 is configured to be implanted temporarily within a body of a patient and/or is configured to be removed (e.g., explanted) from the patient's body after a period of time. In such instances, it may be preferable for substrate 22 to remain within the patient's body and be composed biocompatible materials that are non-biodegradable or non-bioresorbable. Example biocompatible materials may include, but are not limited to, polypropylene, polyester, polytetrafluoroethylene, polyamides, silicones, polysulfones, metals, alloys, and/or combinations thereof. In some embodiments, the biocompatible non-biodegradable and/or non-bioresorbable material or materials may include polymeric and/or non-polymeric materials, such as, for example, polyurethane, polyester, polytetrafluoroethylene (PTFE), polyethylacrylate/polymethylmethacrylate, polyvinyl chloride, polymeric or silicone rubber, thermoplastics, or combinations thereof.

In some embodiments, substate 22 may be a biodegradable or bioresorbable configured to ensure low complication rates and good aesthetic results. This includes meshes that can withstand low doses radiation as some patients may still need radiation treatment following implantation. Physicians may prefer a substrate 22 that is easy to handle and has good flexibility so that IMD 20 contours with device 25 and tissue. Substrate 22 and IMD 20 flexibility and tear resistance may be controlled, in part, by the weave pattern, the yarn material, mesh porosity, and mesh thickness of substrate 22. Substrate 22 that can preventatively lower the risk of infection and promote fast tissue incorporation and tissue remodeling as well as neovascularization may be advantageous. Neovascularization will ensure that new healthy tissue is being generated in place of scar tissue. PLGA based meshes have a good record of biocompatibility that have shown favorable tissue remodeling results under histology.

In some examples, substrate 22 should be constructed to maintain mechanical properties that can sufficiently hold device 25 during periods of tissue remodeling. Similarly, substrate 22 should be easy to suture and have suture pull out strength so that IMD 20 and device 25 can remain fixed upon implant with minimal movement. Permanent surgical meshes often induce scar tissue formation and excessive tissue fibrosis which can make reintervention more difficult and can reduce the aesthetic appearance. This would be minimized with a fully biodegradable or bioresorbable IMD 20. As such, constructing substrate 22 to be flexible and biodegradable or bioresorbable could help lower the rate of capsular contracture, even with skin sparing mastectomy the substate mesh can support the supplemental tissue and inframammary fold, prevent infection, and enable vascularization. With localized controlled release of chemotherapeutics, radiation therapy can be terminated or minimized, shortening the time for reconstruction procedures.

As shown in FIG. 4, IMD 20 may include a first coating 24 that is applied directly on substrate 22 such that first coating 24 covers all or a portion of substrate 22. First coating 24 includes one or more of therapeutic agents (e.g., active pharmaceutical ingredients (APIs), drugs, adjuvants, or other ingredients intended to produce a specified biological response for therapeutic purposes). More specifically, first coating 24 may include therapeutic agents (e.g., one or more chemotherapeutic agents) intended to be released over a prolong period of time. Suitable chemotherapeutic agents that may be used in first coating 24 may include, for example, paclitaxel, doxorubicin, cisplatin, combinations thereof and the like. The polymer of first coating 24 can include one or more of the biodegradable or bioresorbable polymers configured to help control the release of the chemotherapeutic agent over a relatively long period of time (e.g., over a period of at least 10 days to about 180 days) to provide targeted continuous application of the chemotherapeutic agent to the treatment site for an extended period. Such targeted delivery of chemotherapeutic agents can be advantageous compared to conventional chemotherapy treatments that typically administer increased concentrations of such agents in independent treatments leading to concentration variances and compliance challenges. In some examples, due to the arrangement of second coating 26 being on first coating 24, the release characteristics of first coating 24, may be temporarily delayed as second coating 26 begins to degrade. Such a delay may be advantageous in examples where the outermost coating (e.g., second coating 26) includes one or more antibiotic agents to allow the treatment site to briefly heal before the administration of the first therapeutic agent (e.g., chemotherapeutic agent). For example, first coating 24 may have a delayed release similar to the time period it takes for the second coating 26 to substantially degrade. In some examples, the delay before release of the first coating 24 may be about 1-21 days, 3-14 days, or 7-14 days. It is envisioned that IMD 20 can be configured to release the chemotherapeutic agents from first coating 24 after rifampin and/or minocycline are released from second coating 26 (e.g., sequential release), at the same time that rifampin and/or minocycline are released from second coating 26 (e.g., simultaneous release), or both. Additionally, or alternatively, the release profile of first coating 24 containing such chemotherapeutic agents may vary over time such as being slow initially while second coating 26 is present and increase once second coating 26 has degraded.

In some embodiments, first coating 24 is applied to substrate 22 such that coating 24 directly engages fibers, such as, for example, fibers F that form substrate 22 such that coating 24 extends 360 degrees about each of fibers F along at least a portion of a length of each of fibers F, as shown in FIG. 4, for example. That is, fibers F each have a length extending along a longitudinal axis LA and coating 24 extends 360 degrees about axis LA of each fiber F. While fiber F is depicted as a monofilament structure in FIG. 4, in some examples, fiber F may be a multifilament (e.g., yarn) structure. In some embodiments, coating 24 extends 360 degrees about each of fibers F along the entire length of each of fibers F. In some embodiments, coating 24 extends less than 360 degrees about each of fibers F along the entire length of each of fibers F such that coating 24 does not completely surround fibers F. That is, in some embodiments, at least a portion of fibers F is not coated by coating 24.

In some embodiments, first coating 24 has a thickness T1 that is defined by the distance from an inner portion 24a of coating 24 that directly engages at least one of fibers F to an outer portion 24b of coating 24 that is opposite portion 24a. In some thickness T1 may be substantially uniform (e.g., uniform or nearly uniform) 360 degrees about each of fibers F along a length of each of fibers F. In some embodiments, thickness T1 is variable 360 degrees about each of fibers F along a length of each of fibers F. That is, coating 24 may be selectively applied to substrate 22 such that fibers F include more or less of coating 24 at different portions along the length of each of fibers F. For example, the described coatings may be applied (e.g., sprayed) onto one side of substrate 22 resulting in a thicker application of the respective coating on that side. In some embodiments, coating 24 is applied to substrate 22 such that coating 24 at least partially coats filaments or fibers of substrate 22, as shown in FIG. 4, without filling apertures 28 of substrate 22 (e.g., FIG. 7). That is, thickness T1 of coating 24 defines gaps 30 between adjacent filaments or fibers F of substrate 22.

IMD 20 of FIG. 4 also includes second coating 26 applied on first coating 24 such that coating 26 covers all or a portion of first coating 24 and substrate 22. In some embodiments, coating 26 includes a polymer and a second therapeutic agent dispersed in the polymer of coating 26 such that the polymer of coating 26 releases the therapeutic agent of coating 26 as the polymer of coating 26 degrades. In some embodiments, the second therapeutic agent may include one or more antibiotics, such as, for example, rifampin and minocycline. In some embodiments, the therapeutic agent of coating 26 can include a mixture of rifampin and minocycline. In some embodiments, the polymer of coating 26 can include one or more of the polymers discussed herein configured to administer the one or more antibiotics over an intermediate period of time. For example, in some embodiments, second coating 26 may be configured to release the second therapeutic agent (e.g., antibiotic(s)) over a period of about 1 to about 180 days or the lifespan of fibers F following implantation of IMD 20, more preferably over a duration of at least about 3 to at least about 10 days following the initial implantation of IMD 20. Having second coating 26 release the one or more antibiotics over the course of at least about 3 to 10 days may be beneficial to the wound health of the implantation site and greatly reduce the risk of post procedure infection.

Any suitable antibiotic agent may be used in second coating 26 provided the coating is configured to release the antibiotic agent at a minimally effective amount over the specified duration of time (e.g., at least 3 days). Examples of antibacterial agents that may be used include, but are not limited to, triclosan, chlorohexidine and other cationic biguanides, rifampin, minocycline (or other tetracycline derivatives), vancomycin, gentamycin; gendine; genlenol; genfoctol; clofoctol; cephalosporins and the like. Further antibacterial agents or antimicrobials include aztreonam; cefotetan and its disodium salt; loracarbef; cefoxitin and its sodium salt; cefazolin and its sodium salt; cefaclor; ceftibuten and its sodium salt; ceftizoxime; ceftizoxime sodium salt; cefoperazone and its sodium salt; cefuroxime and its sodium salt; cefuroxime axetil; cefprozil; ceftazidime; cefotaxime and its sodium salt; cefadroxil; ceftazidime and its sodium salt; cephalexin; hexachlorophene; cefamandole nafate; cefepime and its hydrochloride, sulfate, and phosphate salt; cefdinir and its sodium salt; ceftriaxone and its sodium salt; cefixime and its sodium salt; cetylpyridinium chloride; ofoxacin; linexolid; temafloxacin; fleroxacin; enoxacin; gemifloxacin; lomefloxacin; astreonam; tosufloxacin; clinafloxacin; cefpodoxime proxetil; chloroxylenol; methylene chloride, iodine and iodophores (povidone-iodine); nitrofurazone; meropenem and its sodium salt; imipenem and its sodium salt; cilastatin and its sodium salt; azithromycin; clarithromycin; dirithromycin; erythromycin and hydrochloride, sulfate, or phosphate salts ethylsuccinate, and stearate forms thereof, clindamycin; clindamycin hydrochloride, sulfate, or phosphate salt; lincomycin and hydrochloride, sulfate, or phosphate salt thereof, tobramycin and its hydrochloride, sulfate, or phosphate salt; streptomycin and its hydrochloride, sulfate, or phosphate salt; vancomycin and its hydrochloride, sulfate, or phosphate salt; neomycin and its hydrochloride, sulfate, or phosphate salt; acetyl sulfisoxazole; colistimethate and its sodium salt; quinupristin; dalfopristin; amoxicillin; ampicillin and its sodium salt; clavulanic acid and its sodium or potassium salt; penicillin G; penicillin G benzathine, or procaine salt; penicillin G sodium or potassium salt; carbenicillin and its disodium or indanyl disodium salt; piperacillin and its sodium salt; α-terpincol; thymol; taurinamides; nitrofurantoin; silver-sulfadiazine; hexetidine; methenamine; aldehydes; azylic acid; silver; benzyl peroxide; alcohols; carboxylic acids; salts; nafcillin; ticarcillin and its disodium salt; sulbactam and its sodium salt; methylisothiazolone, moxifloxacin; amifloxacin; pefloxacin; nystatin; carbepenems; lipoic acids and its derivatives; beta-lactams antibiotics; monobactams; aminoglycosides; microlides; lincosamides; glycopeptides; tetracyclines; chloramphenicol; quinolones; fucidines; sulfonamides; macrolides; ciprofloxacin; ofloxacin; levofloxacins; teicoplanin; mupirocin; norfloxacin; sparfloxacin; ketolides; polyenes; azoles; penicillins; echinocandins; nalidixic acid; rifamycins; oxalines; streptogramins; lipopeptides; gatifloxacin; trovafloxacin mesylate; alatrofloxacin mesylate; trimethoprims; sulfamethoxazole; demeclocycline and its hydrochloride, sulfate, or phosphate salt; doxycycline and its hydrochloride, sulfate, or phosphate salt; minocycline and its hydrochloride, sulfate, or phosphate salt; tetracycline and its hydrochloride, sulfate, or phosphate salt; oxytetracycline and its hydrochloride, sulfate, or phosphate salt; chlortetracycline and its hydrochloride, sulfate, or phosphate salt; metronidazole; dapsone; atovaquone; rifabutin; linezolide; polymyxin B and its hydrochloride, sulfate, or phosphate salt; sulfacetamide and its sodium salt; and clarithromycin (and combinations thereof). Synthetic mimics of antimicrobial peptides or naturally derived antimicrobial peptides are also examples of antimicrobial agents could be included in second coating 26. In some embodiments second coating 26 may contain rifampin and another antimicrobial agent, such as, for example, an antimicrobial agent that is a tetracycline derivative. In some embodiments, second coating 26 contains a cephalosporin and another antimicrobial agent. In some embodiments, second coating 26 contains combinations including rifampin and minocycline, rifampin and gentamycin, and rifampin and minocycline. Examples of other antimicrobials include amphotericin B; pyrimethamine; flucytosine; caspofungin acetate; fluconazole; griseofulvin; terbinafine and its hydrochloride, sulfate, or phosphate salt; amorolfine; triazoles (Voriconazole); flutrimazole; cilofungin; LY303366 (echinocandins); pneumocandin; imidazoles; omoconazole; terconazole; fluconazole; amphotericin B, nystatin, natamycin, liposomal amptericin B, liposomal nystatins; griscofulvin; BF-796; MTCH 24; BTG-137586; RMP-7/Amphotericin B; pradimicins; benanomicin; ambisome; ABLC; ABCD; Nikkomycin Z; flucytosine; SCH 56592; ER30346; UK 9746; UK 9751; T 8581; LY121019; ketoconazole; micronazole; clotrimazole; econazole; ciclopirox; naftifine; and itraconazole. In preferred examples, second coating 26 may include a combination of at least rifampin and minocycline. Rifampin and minocycline may be combined at a ratio ranging from about 10:1 to about 1:10, about 5:2 to about 2:5, or about 1:1.

Second coating 26 may be applied on but spaced apart from substrate 22 by coating 24, as shown in FIG. 4, for example. In some embodiments, second coating 26 partially encapsulates first coating 24 (as shown in FIG. 4). For example, second coating 26 may directly engage first coating 24 such that coating 26 extends less than 360 degrees about each of fibers F along at least a portion of the length of each of fibers F. That is, coating 26 extends less 360 than degrees about axis LA of each fiber F. In some embodiments, coating 26 extends only between about 150 degrees and about 330 degrees about axis LA of each fiber F. In some embodiments, coating 26 extends only between about 170 degrees and about 310 degrees about axis LA of each fiber F. In some embodiments, coating 26 extends only between about 190 degrees and about 290 degrees about axis LA of each fiber F. In some embodiments, coating 26 extends only between about 210 degrees and about 270 degrees about axis LA of each fiber F. In some embodiments, coating 26 extends only between about 230 degrees and about 250 degrees about axis LA of each fiber F. In some embodiments, second coating 26 extends only about 240 degrees about axis LA of each fiber F.

Second coating 26 may applied to first coating 24 and substrate 22 such that the apertures (e.g., apertures 28) created by substrate 22 remain open or are occluded. For example, as shown in FIGS. 3 and 7, second coating 26 covers or fills the apertures between adjacent fibers F (e.g., apertures 28 and 30 shown in FIGS. 6 and 7). The filled gaps 30 would then become uncovered and/or unfilled post-implantation as coating 26 degrades. Apertures 30 are then exposed as second coating 26 degrades providing greater exposure of first coating 24 to the surrounding tissue and fluids. Alternatively, second coating 26 and first coating 24 may only cover partial of the entire mesh leaving apertures 30 open at initial implantation.

Alternatively, second coating 26 and first coating 24 may each cover only portions of substrate 22, thereby avoiding potential drug interactions between coating 24 and coating 26. For example, in some embodiments, coating 24 is spaced apart from coating 26 each within distinct regions of substrate 22. In some embodiments, coating 24 coats a first portion of substrate 22 and coating 26 coats a second portion of substrate 22 that is spaced apart from the first portion of substrate. In some embodiments, coating 24 does not overlap coating 26, and vice versa.

As shown in FIG. 4, medical IMD 20 defines a first major surface 32 and an opposite second major surface 34. Each surface 32 and 34 may present with different coating characteristics. For example, second coating 26 may solely define surface 32 and a combination of first and second coatings 24, 26 may define surface 34, as shown in FIG. 4. The configuration of surface 32 and surface 34 allows IMD 20 to be selectively oriented and implanted in a patient for targeted release characteristics to the adjacent tissue. For example, surface 32 may be directed toward patient tissue such that surface 32, and hence second coating 26, preferentially degrades and releases one or more antibiotic agents, such as, for example, rifampin and/or minocycline and/or other therapeutic agents from second coating 26 upon contact over a shorter duration of time, with surface 34 oriented toward medical device 25. Additionally, or alternatively, surface 34 may be positioned toward patient tissue for more immediate degradation of said surface and of first and second coatings 24 and 26, with surface 34 oriented toward medical device 25. In examples, where medical device 25 is not present, the respective surfaces 32 and 34 may each contact surrounding tissue and respectively disperse the therapeutic agents to the adjacent tissue.

Surface 32 and/or surface 34 may be characterized as planar such that surface 32 extends parallel to surface 34. In some embodiments, one or more of fibers F extends through surface 32 and/or surface 34. In some embodiments, one or more of fibers F extends through surface 34 without extending through surface 34. In some embodiments, coating 24 directly engages and/or is continuous with surface 34 and coating 24 is spaced apart from surface 32. In some embodiments, fibers F are spaced apart from surface 32 by coating 24 and coating 26. In some embodiments, thickness T1 is greater than or equal to a thickness T2 of coating 26 that is defined by the distance from surface 32 to surface 34. In some embodiments, thickness T2 is greater than thickness T1. In some embodiments, thickness T2 is at least 1.5 times greater than thickness T1. In some embodiments, thickness T2 is at least 2.0 times greater than thickness T1. In some embodiments, thickness T2 is at least 2.5 times greater than thickness T1. In some embodiments, thickness T2 is at least 3.0 times greater than thickness T1. In some embodiments, thickness T2 is at least 3.5 times greater than thickness T1. In some embodiments, thickness T2 is at least 4.0 times greater than thickness T1. In some embodiments, thickness T2 is at least 4.5 times greater than thickness T1. In some embodiments, thickness T2 is at least 5.0 times greater than thickness T1. In some embodiments, thickness T2 more than 5.0 times greater than thickness T1. In some embodiments, first coating 24 and/or second coating 26 may be selectively applied to substrate 22 such that coating 24, coating 26, or both are thicker on one side of substrate 22 than on another side of substrate 22. For example, first coating 24 may be applied (e.g., sprayed, spun, or the like) to a first side of substrate 22 followed by second coating 26 being applied to a second side of substrate 22 such that each side of IMD 20 has different therapeutic coating characteristics.

The polymers used for the formation of first and second coatings 24 and 26 should be selected such that they do not interfere with the activity of the therapeutic agents dispersed therein, while also degrading over a specified period of time to obtain the desired release profiles of the therapeutic agents. Some suitable carrier polymers may include, but are not limited to biodegradable polymers such as polylactic acid, polyglycolic acid, poly(L-lactide), poly(D,L-lactide) polyglycolic acid[polyglycolide], poly(L-lactide-co-D,L-lactide), poly(L-lactide-co-glycolide), poly(D, L-lactide-co-glycolide), poly(glycolide-co-trimethylene carbonate), poly(D,L-lactide-co-caprolactone), poly(glycolide-co-caprolactone), polyethylene oxide, polydioxanone, polypropylene fumarate, poly(glutamic acid), poly(ethyl glutamate-co-glutamic acid), poly(tert-butyloxy-carbonylmethyl glutamate), polycaprolactones (PCL, e.g., ε-caprolactone), polycaprolactone co-butylacrylate, polyhydroxybutyrate, copolymers of polyhydroxybutyrate, poly(phosphazene), poly(phosphate ester), polyamides (e.g., poly(amino acids), polydepsipeptides, maleic anhydride copolymers, polycarbonates, polyiminocarbonates, poly[(97.5% dimethyl-trimethylene carbonate)-co-(2.5% trimethylene carbonate)], poly(orthoesters), tyrosine-derived polyarylates, tyrosine-derived polycarbonates, tyrosine-derived polyiminocarbonates, tyrosine-derived polyphosphonates, polyethylene oxide, polyethylene glycol, copolymer polyesters (e.g., poly(lactic-co-glycolic acid) (PLGA), poly(alkyl cyanoacrylates) (PACAs)), poly(L-lysine), polyalkylene oxides, polyethylene glycol (PEG), hydroxypropylmethylcellulose, BTE glutarate, DTM glutarate, DT propylamide glutarate, DT glycineamide glutarate, BTE succinate, BTM succinate, BTE succinate PEG, BTM succinate PEG, DTM succinate PEG, DTM succinate, DT N-hydroxysuccinimide succinate, DT glucosamine succinate, DT glucosamine glutarate, DT PEG ester succinate, DT PEG amide succinate, DT PEG ester glutarate, DT PEG ester succinate, DTMB P (Desaminotyrsoyl tyrosine methylparaben ester—glutarate), and DTPP P (Desaminotyrsoyl tyrosine propylparaben ester—glutarate), polysaccharides such as hyaluronic acid, chitosan, regenerate cellulose, combinations, blends, mixtures, and block co-polymers of thereof.

PLGA polymers with higher lactic acid content tend to be more hydrophobic favoring a longer degradation rate and longer release profile. In some examples, PCL can be blended or synthesized as a block polymer with PLGA to tune the drug release rate. The degradation rate of polyamides may be dependent on the hydrophilicity of the amino acids that make up the polymer. In some examples, water soluable polymers may be used including, but not limited to, poly(ethylene glycol) PEG, and poly(N-vinyl pyrrolidon), and poly(vinyl alcohol). Because these are water soluble, such polymers tend to release drug in a more diffusion-controlled manner or through a solvent-activated (swelling) mechanism. Such polymers can be blended, copolymerized, or be used with different molecular weights to tailor the release profile.

In some embodiments, the carrier polymer (e.g., second coating 26) is a polyarylate such as a tyrosine-derived polyarylate. In some embodiments, the tyrosine-derived polyarylate is p (DTE co X % DT succinate), where X is about 10% to about 30%. In some embodiments, the tyrosine-derived polyarylate is p (DTE co X % DT succinate), where X ranges from about 26.5% to about 28.5%. In some embodiments, the tyrosine-derived polyarylate is p (DTE co X % DT succinate), where X is about 27.5%. In some embodiments, the polymer is P22-27.5 DT. As used herein, DTE is the diphenol monomer desaminotyrosyl-tyrosine ethyl ester; DTBn is the diphenol monomer desaminotyrosyl-tyrosine benzyl ester; DT is the corresponding free acid form, namely desaminotyrosyl-tyrosine. BTE is the diphenol monomer 4-hydroxy benzoic acid-tyrosyl ethyl ester; BT is the corresponding free acid form, namely 4-hydroxy benzoic acid-tyrosinc. P22-XX is a polyarylate copolymer produced by condensation of DTE and DTBn with succinic acid followed by removal of benzyl group. P22-10, P22-15, P22-20, P22-XX, etc., represents copolymers different percentage of DT (i.e., 10, 15, 20 and % DT, etc.) In some embodiments, the polymer is produced by condensation of DTBn with succinic acid followed by removal of benzyl group. This polymer is represented as P02-100. In some embodiments, the carrier polymer includes one or more polyarylates that are copolymers of desaminotyrosyl-tyrosine (DT) and an desaminotyrosyl-tyrosyl ester (DT ester), wherein the copolymer comprises from about 0.001% DT to about 80% DT and the ester moiety can be a branched or unbranched alkyl, alkylaryl, or alkylene ether group having up to 18 carbon atoms, any group of which can, optionally have a polyalkylene oxide therein. Similarly, another group of polyarylates are the same as the foregoing but the desaminotyrosyl moiety is replaced by a 4-hydroxybenzoyl moiety. In some embodiments, the DT or BT contents include those copolymers with from about 1% to about 30%, from about 5% to about 30% from about 10% to about 30% DT or BT. In some embodiments, the diacids (used informing the polyarylates) include succinate, glutarate and glycolic acid.

In some embodiments, the polymer of coating 24 and/or the polymer of coating 26 is one more polymers from the DTE-DT succinate family of polymers, e.g., the P22-xx family of polymers having from 0-50%, 5-50%, 5-40%, 1-30% or 10-30% DT, including but not limited to, about 1, 2, 5, 10, 15, 20, 25, 27.5, 30, 35, 40%, 45% and 50% DT. In some embodiments, the polymer is P22-27.5 DT.

In some embodiments, the polymer of coating 24 and/or the polymer of coating 26 can have from 0.1-99.9% PEG diacid to promote the degradation process. In some embodiments, the polymer of coating 24 and/or the polymer of coating 26 includes blends of polyarylates or other biodegradable polymers with polyarylates.

In some embodiments, the polymer of first coating 24 may include poly(lactic-co-glycolic acid, polycaprolactones, polyvinylethers, polyphosphazenes, poly(alkyl cyanoacrylates), poly(amino acids), a tyrosine-derived polyarylate, collagen, alginate, chitosan, hyaluronic acid, polyhydroxyalkanoates (PHA), starch, cellulose, or a combination thereof and the polymer of second coating 26 can include a tyrosine-derived polyarylate, such as, for example, one or more of the tyrosine-derived polyarylates discussed herein.

The drug release characteristics of the described layers from a respective polymer may be adjusted by controlling certain parameters of the polymer such as crystallinity, glass transition temperature, polymer molecular weight, polymer hydrophobicity, and solubility of the drug in the polymer system. Crystallinity can affect the macro properties of the polymer including its ability to swell and undergo hydrolysis. In some examples, a high degree of crystallinity results in a slower drug release as it is harder for water to diffuse into the polymer system and for the drug to diffuse out. The glass transition temperature dictates whether the amorphous region of the polymer is in a “glassy” or “rubbery” state. Below the glass transition the polymer is in a glassy state and the incorporated drug will have low mobility and low diffusion rates. Above glass transition temperature the polymer is rubbery which enables higher mass transfer. Polymer molecular weight affects the hydrophobicity of the system as well as porosity of the system. Polymers with higher molecular weights tend to be more hydrophobic resisting water ingress more easily than low molecular weight polymers. Solubility is dependent on the chemical nature of the drug and the side chains of the polymer. Polymers that have more favorable molecular interactions (e.g., hydrogen bonding, electrostatic) with the drug will be able to retain the drug more easily than other polymers.

In some embodiments, substrate 22 may incorporate one or more of the above describe biodegradable or bioresorbable polymers and therapeutic agents. For example, fibers F used to produce substrate 22 may be embedded with a biodegradable or bioresorbable polymer and a chemotherapeutic agent (not shown in FIG. 4). In such examples, the incorporated chemotherapeutic material (e.g., a chemotherapeutic agent and polymer carrier) may be added in addition to first coating 24 to provide additional release of a chemotherapeutic agent over time as substrate 22 degrades within the body. The embedded polymer carrier and chemotherapeutic agent may be the same or different than the material use in first coating 24.

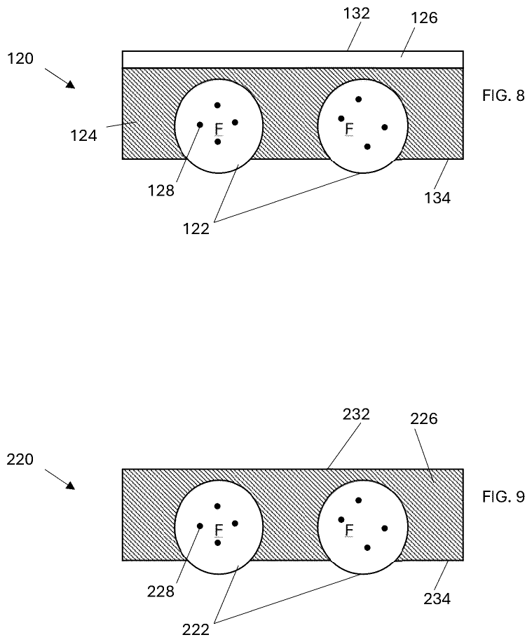

FIG. 8 illustrates a cross-sectional view on another embodiment of a biodegradable or bioresorbable medical device 120 that includes substrate 122 having fibers F, chemotherapeutic layer 124 (e.g., polymeric coating layer containing one or more chemotherapeutic agents substantially similar to first coating 24 of FIG. 4) applied directly to fibers F such that layer 124 covers at least a portion of fibers F and antibiotic layer 126 (e.g., polymeric coating layer containing one or more antibiotic agents substantially similar to second layer 26 of FIG. 4) is applied to cover a portion of chemotherapeutic layer 124. That is, antibiotic layer 124 forms surface 132 of implantable medical device 120 and chemotherapeutic layer 124 forms surface 134 of device 120 such that device 120 can be implanted with surface 132 directly engaging surrounding tissue so that antibiotic layer 126 directly contacts tissue to promote wound health with layer 134 oriented toward implantable medical device 25 (if present) or additional adjacent tissue. Alternatively, surface 134 may be oriented to directly engage surrounding tissue so that chemotherapeutic layer 124 directly contacts tissue cancerous treatment such that coating 124 releases chemotherapeutic agents to the tissue with layer 126 oriented toward implantable medical device 25.

Substrate 122 further includes chemotherapeutic material 128 embedded within fibers F of substrate 122. The embedded chemotherapeutic material may include a polymeric carrier and one or more chemotherapeutic agents such as the same similar material to chemotherapeutic layer 124. Due to the embedded nature of material 128, the chemotherapeutic agent of the material may release as fibers F degrade.

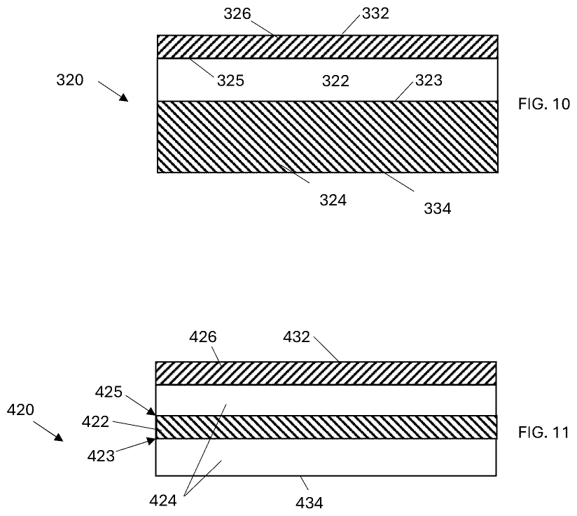

FIG. 9 illustrates a cross-sectional view on another embodiment of a biodegradable or bioresorbable medical device 220 that includes substrate 222 having fibers F with chemotherapeutic material 228 (e.g., polymeric carrier contain one or more chemotherapeutic agents substantially similar to first coating 24 of FIG. 4) embedded therein and antibiotic layer 226 (e.g., polymeric coating layer containing one or more antibiotic agents substantially similar to second layer 26 of FIG. 4) applied to substrate 222. That is, antibiotic layer 224 forms surface 232 of implantable medical device 220 and a portion of surface 234 of device 220. As device 220 degrades, antibiotic layer 224 releases the one or more antibiotic agents over a relatively short period of time (e.g., 3-10 days) while the chemotherapeutic agent is released over a prolonged period of time (e.g., 10-180 days) as substrate 222 and the carrier polymer of chemotherapeutic material 228 degrade.

FIGS. 10 and 11 illustrate additional embodiments for IMD 20 with other possible layer constructions and arrangements. FIG. 10 illustrates a cross-section of an example IMD 320 that includes substrate 322 (e.g., substantially similar to substrate 22) with chemotherapeutic layer 324 (e.g., polymeric coating layer containing one or more chemotherapeutic agents substantially similar to first coating 24 of FIG. 4) applied directly to first side 323 of substrate 322 such that chemotherapeutic layer 324 sets on at least a portion of substrate 322 and forms exterior surface 334. Antibiotic layer 326 (e.g., polymeric coating layer containing one or more antibiotic agents substantially similar to second layer 26 of FIG. 4) is applied to a second side 325 of substrate 322 such that antibiotic layer 326 sets on at least a portion of substrate 322 and forms exterior surface 332. That is, antibiotic layer 324 forms surface 332 of implantable medical device 320 and chemotherapeutic layer 324 forms surface 334 of device 320 such that device 320 can be implanted with surface 332 directly engaging surrounding tissue so that antibiotic layer 326 directly contacts tissue to promote wound health with chemotherapeutic layer 324 oriented toward implantable medical device 25 (if present) or additional adjacent tissue.

FIG. 11 illustrates a cross-section of another example IMD 420 that includes substrate 422 (e.g., substantially similar to substrate 22) with chemotherapeutic layer 424 (e.g., polymeric coating layer containing one or more chemotherapeutic agents substantially similar to first coating 24 of FIG. 4) applied directly to both first side 423 and second side 425 of substrate 422 such that chemotherapeutic layer 424 forms exterior surface 434. Antibiotic layer 426 (e.g., polymeric coating layer containing one or more antibiotic agents substantially similar to second layer 26 of FIG. 4) is then applied to one side of substrate 422 (e.g., second side 425) such that antibiotic layer 426 sets on at least a portion of chemotherapeutic layer 424 and forms exterior surface 432. That is, antibiotic layer 424 forms surface 432 of implantable medical device 420 and chemotherapeutic layer 424 forms surface 434 of device 420 such that device 420 can be implanted with surface 432 directly engaging surrounding tissue so that antibiotic layer 426 directly contacts tissue to promote wound health with chemotherapeutic layer 424 oriented toward implantable medical device 25 (if present) or additional adjacent tissue. The presence of a portion of chemotherapeutic layer 424 directly between substrate 422 and antibiotic layer 436 may delay the release of the chemotherapeutic agent from this portion.

In some embodiments, one or more of the above described coatings or layers (e.g., first and second coatings 24 and 26) can include one or more additional therapeutic agents in addition to the chemotherapeutic and antibiotic agents discussed above including, but not limited to, one or more of the hemostatic agents, one or more anesthetics, one or more antibiotics, one or more anti-inflammatory agent, one or more procoagulant agent, one or more fibrosis-inhibiting agent, one or more anti-scarring agent, one or more antiseptics, one or more leukotriene inhibitors/antagonists, one or more cell growth inhibitors and mixtures thereof. The hemostatic agent may be included in one of the described coatings (e.g., second coating 26) to assist with immediate bleed control and promote hemostasis after implantation. Example hemostatic agents may include tranexamic acid, epinephrine, ZnCl2 (Mohn paste), alginate, chitosan, collagen (e.g., acid soluble collagen, pepsin soluble collagen, gelatin, cross-linkable collagen, or fibrillar collagen), oxidized regenerated cellulose, ellagic acid (e.g., naturally derived polyphenol ellagic acid), Spongostan®, Surgifoam®, Avitene, thrombin and Ostene®, protamine, norepinephrine, desmopressin, lysine analogs, gelatin, polysaccharide spheres, mineral zeolite, bovine thrombin, pooled human thrombin, recombinant thrombin, gelatin and thrombin, collagen and thrombin, cyanacrylate, fibrin glue, polyethylene glycol, glutaraldehyde, or combinations thereof. In some examples, using ellagic acid in coating can be beneficial due to the hemostatic, antioxidant, anti-proliferative and wound healing properties of ellagic acid.

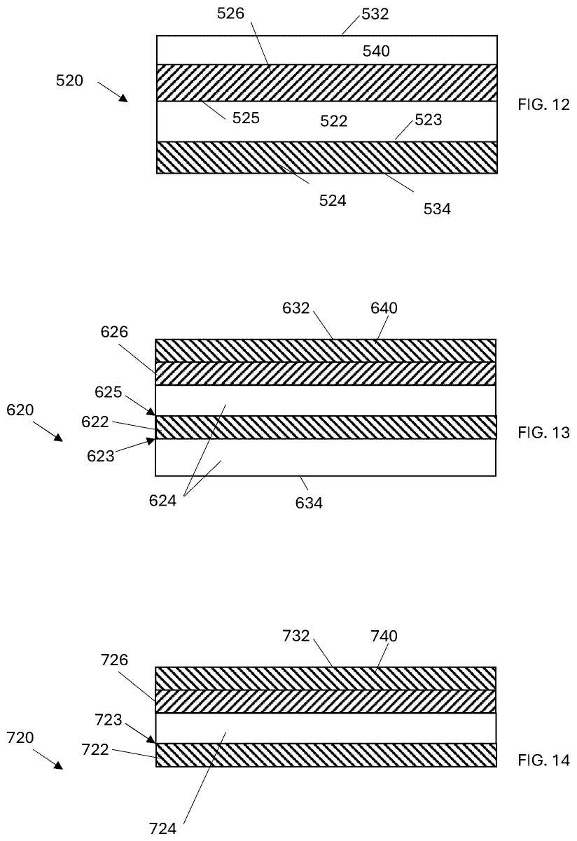

Additionally, or alternatively, it may be beneficial include a hemostatic layer (e.g., carrier polymer with a hemostatic agent dispersed in the carrier polymer) in any of the above IMDs (IMD 20, 120, 220, 320, and 420). For example, the hemostatic agent may be dispersed in a carrier polymer, such as collagen or alginate and applied to one or more of the outer surfaces of the above IMDs, configured to release the hemostatic agent over a relatively short period of time (e.g., less than 3 days) to provide near immediate bleed control properties to one or more of the above IMDs. FIGS. 12-14 provide various examples of layer configurations of how the described hemostatic layer may be incorporated into the describe IMDs.

FIG. 12 illustrates a cross-section of an example IMD 520 that includes substrate 522 (e.g., substantially similar to substrate 22) with chemotherapeutic layer 524 (e.g., polymeric coating layer containing one or more chemotherapeutic agents substantially similar to first coating 24 of FIG. 4) applied directly to first side 523 of substrate 522 such that chemotherapeutic layer 524 sets on at least a portion of substrate 522 and forms exterior surface 534. Antibiotic layer 526 (e.g., polymeric coating layer containing one or more antibiotic agents substantially similar to second layer 26 of FIG. 4) is applied to a second side 525 of substrate 522 such that antibiotic layer 526 sets on at least a portion of substrate 522. Hemostatic layer 540 (e.g., polymeric coating layer containing one or more hemostatic agents as described above) is applied to on antibiotic layer 526 such that hemostatic layer 540 sets on antibiotic layer 526 and forms exterior surface 532. That is, hemostatic layer 540 forms surface 532 of implantable medical device 520 and chemotherapeutic layer 524 forms surface 534 of device 520 such that device 520 can be implanted with surface 532 directly engaging surrounding tissue so that hemostatic layer 540 directly contacts tissue to promote bleed control followed by release of the antibiotic agent from layer 526 once layer 540 sufficiently degrades.

FIG. 13 illustrates a cross-section of another example IMD 620 that includes substrate 622 (e.g., substantially similar to substrate 22) with chemotherapeutic layer 624 (e.g., polymeric coating layer containing one or more chemotherapeutic agents substantially similar to first coating 24 of FIG. 4) applied directly to both first side 623 and second side 625 of substrate 622 such that chemotherapeutic layer 624 forms exterior surface 634. Antibiotic layer 626 (e.g., polymeric coating layer containing one or more antibiotic agents substantially similar to second layer 26 of FIG. 4) is then applied to one side of substrate 622 (e.g., second side 625) such that antibiotic layer 626 sets on at least a portion of chemotherapeutic layer 624. Hemostatic layer 640 (e.g., polymeric coating layer containing one or more hemostatic agents as described above) is then applied to one side of substrate 622 (e.g., second side 625) such that hemostatic layer 640 sets on at least a portion of antibiotic layer 626 or chemotherapeutic layer 624). As shown in FIG. 13, hemostatic layer 640 forms surface 632 of implantable medical device 620 and chemotherapeutic layer 624 forms surface 634 of device 620 such that device 620 can be implanted with surface 632 directly engaging surrounding tissue.

FIG. 14 illustrates a cross-section of an example IMD 720 that includes substrate 722 (e.g., substantially similar to substrate 22) with chemotherapeutic layer 724 (e.g., polymeric coating layer containing one or more chemotherapeutic agents substantially similar to first coating 24 of FIG. 4) applied directly to first side 723 of substrate 722 such that chemotherapeutic layer 724 sets on at least a portion of substrate 722. Antibiotic layer 726 (e.g., polymeric coating layer containing one or more antibiotic agents substantially similar to second layer 26 of FIG. 4) is applied on chemotherapeutic layer 724. Hemostatic layer 740 (e.g., polymeric coating layer containing one or more hemostatic agents as described above) is applied to on antibiotic layer 726 such that hemostatic layer 740 sets on antibiotic layer 726 and forms exterior surface 732.

One or more portions of the above described IMD 20 may be prepared using following examples. Chemotherapeutic Coating Example 1. A chemotherapeutic coating (e.g., first coating 24) is applied to a biodegradable copolymer mesh of glycolide and lactide substrate (e.g., substrate 22). The coating is prepared by adding about 200 mL of distilled water to 2.65 g of glycerol, 6.6 g of collagen, DMSO and 50 μg of paclitaxel (chemotherapeutic agent) to form a solution S1. Solution S1 is heated to 40° C. and then to 65° C. As solution S1 is heated from 40° C. to 65° C., the pH of solution S1 is adjusted to about 7.0, until the collagen is fully dissolved. Solution S1 is then poured into a 31 cm by 38 cm mold. Substrate 22 is added to a top layer of solution S1 and is then removed from solution S1 and air dried overnight. The air-drying forms a dried coated mesh F1. In some embodiments, coated mesh F1 is vacuum dried for 4 to 8 hours.

Chemotherapeutic Coating Example 2. A chemotherapeutic coating (e.g., first coating 24) is applied to a biodegradable copolymer mesh of glycolide and lactide substrate (e.g., substrate 22). The coating is prepared by adding about 200 mL of distilled water to 2.65 g of glycerol, 6.6 g of collagen, DMSO and 10 mM of doxorubicin (chemotherapeutic agent) to form a Solution S2. Solution S2 is heated to 40° C. and then to 65° C. As solution S2 is heated from 40° C. to 65° C., the pH of solution S2 is adjusted to about 7.0, until the collagen is fully dissolved. Solution S2 is then poured into a 31 cm by 38 cm mold. Substrate 22 is added to a top layer of solution S2 and is then removed from Solution S2 and air dried overnight. The air-drying forms a dried coated mesh F2. In some embodiments, coated mesh F2 is vacuum dried for 4 to 8 hours.

Chemotherapeutic Coating Example 3. A chemotherapeutic coating (e.g., first coating 24) is applied to a biodegradable copolymer mesh of glycolide and lactide substrate (e.g., substrate 22). The coating is prepared by adding about 300 mL of distilled water to 2.0 g of glycerol, 3.0 g of alginate, DMSO and 50 μg of paclitaxel (chemotherapeutic agent) to form a solution S3. Solution S3 is heated to 40° C. and then to 65° C. As solution S3 is heated from 40° C. to 65° C., the pH of solution S3 is adjusted to about 7.0, until the collagen is fully dissolved. Solution S3 is then poured into a 31 cm by 38 cm mold. Substrate 22 is added to Solution S3 and is then removed from Solution S3 and air dried overnight. The air-drying forms a dried coated mesh F3. In some embodiments, coated mesh F3 is vacuum dried for 4 to 8 hours.

Chemotherapeutic Coating Example 4. A chemotherapeutic coating (e.g., first coating 24) is applied to a biodegradable copolymer mesh of glycolide and lactide substrate (e.g., substrate 22). The coating is prepared by adding about 300 mL of distilled water to 2.0 g of glycerol, 3.0 g of alginate, DMSO and 10 mM of doxorubicin (chemotherapeutic agent) to form a solution S4. Solution S4 is heated to 40° C. and then to 65° C. As solution S4 is heated from 40° C. to 65° C., the pH of solution S4 is adjusted to about 7.0, until the collagen is fully dissolved. Solution S4 is then poured into a 31 cm by 38 cm mold. Substrate 22 is added to Solution S4 and is then removed from Solution S4 and air dried overnight. The air-drying forms a dried coated mesh F4. In some embodiments, coated mesh F4 is vacuum dried for 4 to 8 hours.

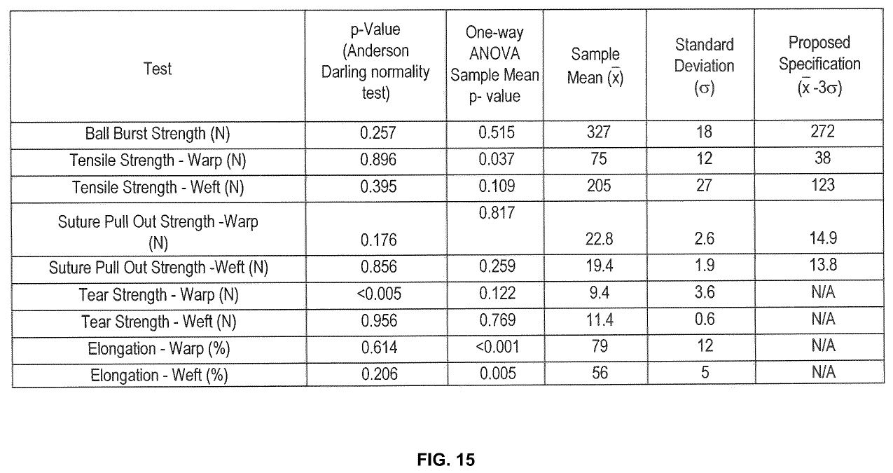

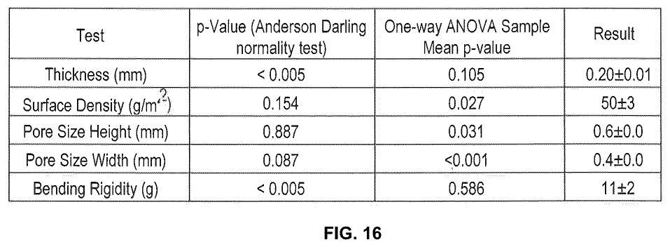

Example 5. Tests for strength and elongation were performed on one embodiment of IMD 20 discussed herein. Results for the tests are shown in FIG. 15. In some embodiments, IMD 20 has a minimum mechanical strength of about 250N, a minimum ball burst strength of about 38-40N, a minimum tensile strength of about 13-15N, a minimum suture pull out strength of about 3-5N. Tests for thickness, surface density, height, width and bending rigidity were performed on the embodiment of IMD 20 discussed regarding FIG. 15. Results for the tests are shown in FIG. 16.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An implantable medical device comprising:
a substrate, wherein the substrate comprises a biodegradable or bioresorbable material;
a first layer on at least a portion of the substrate, the first layer comprising a first biodegradable or bioresorbable polymer and a chemotherapeutic agent dispersed in the first polymer, wherein the first layer is configured to release the chemotherapeutic agent over a first period of time as the first polymer degrades, wherein the first period of time is at least 10 days; and
a second layer on at least a portion of the first coating or the substrate, the second layer comprising a second biodegradable or bioresorbable polymer and at least one antibiotic agent dispersed in the second polymer, wherein the second layer is configured to release the at least one antibiotic agent over a second period of time as the second polymer degrades, wherein the second period of time is at least 1 days.

2. The implantable medical device recited in claim 1, wherein the second polymer comprises a tyrosine-derived polyarylate.

3. The implantable medical device in claim 2, wherein the at least one antibiotic comprises rifampin and minocycline.

4. The implantable medical device in claim 1, wherein the second period of time is between about 3-10 days.

5. The surgical device recited in any one of the preceding claims, wherein the first polymer comprises at least one of poly(lactic-co-glycolic acid, polycaprolactones, poly(alkyl cyanoacrylates), poly(amino acids), collagen, alginate, a tyrosine-derived polyarylate, or a combination thereof.

6. The implantable medical device in claim 1, wherein the chemotherapeutic agent comprises at least one of paclitaxel, doxorubicin, or cisplatin.

7. The implantable medical device in claim 1, wherein the first period of time is between 10 and 180 days.

8. The implantable medical device in claim 1, wherein the first layer is coated on a first side of the substrate such that the first layer is thicker on the first side of the substrate compared to a second side of the substrate opposite of the first side.

9. The implantable medical device in claim 1, wherein the second layer forms at least part of an outer surface of the implantable medical device.

10. The implantable medical device in claim 1, further comprising a third layer on at least a portion of the first layer, the second layer, or the substrate, the third layer comprising a third biodegradable of bioresorbable polymer and a hemostatic agent dispersed in the third polymer, wherein the third coating is configured to release the hemostatic agent over a third period of time as the third polymer degrades.

11. The surgical device recited in claim 10, wherein the third layer forms at least part of the outer surface of the implantable medical device.

12. The implantable medical device in claim 1, wherein the substrate comprises a biodegradable or bioresorbable mesh.

13. A surgical system comprising:
a surgical implant; and
an implantable medical device in form of a sheet or pouch configured to at least partially surround the surgical implant, the implantable medical device comprising:
a substrate, wherein the substrate comprises a biodegradable or bioresorbable material;
a first layer on at least a portion of the substrate, the first layer comprising a first biodegradable or bioresorbable polymer and a chemotherapeutic agent dispersed in the first polymer, wherein the first layer is configured to release the chemotherapeutic agent over a first period of time as the first polymer degrades, wherein the first period of time is at least 10 days; and
a second layer on at least a portion of the first coating or the substrate, the second layer comprising a second biodegradable or bioresorbable polymer and at least one antibiotic agent dispersed in the second polymer, wherein the second layer is configured to release the at least one antibiotic agent over a second period of time as the second polymer degrades, wherein the second period of time is at least 1 days.

14. The surgical system of claim 13, wherein the second polymer comprises a tyrosine-derived polyarylate, and wherein the at least one antibiotic comprises rifampin and minocycline.

15. The surgical system of claim 14, wherein the second period of time is between about 3-10 days, and the first period of time is between about 10-180 days.

16. The surgical system of claim 13, wherein the first polymer comprises at least one of poly(lactic-co-glycolic acid, polycaprolactones, poly(alkyl cyanoacrylates), poly(amino acids), collagen, alginate, a tyrosine-derived polyarylate, or a combination thereof, and wherein the chemotherapeutic agent comprises at least one of paclitaxel, doxorubicin, or cisplatin.

17. The surgical system of claim 13, wherein the first layer is coated on a first side of the substrate that is positioned in contact with the surgical implant and the second layer is coated on a second side of the substrate that is positioned away from the surgical implant.

18. The surgical system of claim 13, further comprising a third layer on at least a portion of the first layer, the second layer, or the substrate, the third layer comprising a third biodegradable of bioresorbable polymer and a hemostatic agent dispersed in the third polymer, wherein the third coating is configured to release the hemostatic agent over a third period of time as the third polymer degrades, and wherein the third layer is coated on a side of the substrate that faces away from the surgical implant such that third layer forms at least part of the outer surface of the implantable medical device.

19. The surgical system of claim 13, wherein the substrate comprises a biodegradable or bioresorbable mesh such that the implantable medical device is fully biodegradable or bioresorbable, and wherein the surgical implant comprises a breast implant.

20. An implantable medical device comprising:
a bioresorbable mesh substrate;
a first coating that covers at least a portion of the substrate, the first coating comprising a first biodegradable or bioresorbable polymer and a chemotherapeutic agent dispersed in the first polymer such that the first polymer releases the chemotherapeutic agent as the first polymer degrades, wherein the first coating is configured to release the chemotherapeutic agent over a period of more than 10 days;
a second coating that covers at least a portion of the first coating, the second coating comprising a second biodegradable or bioresorbable polymer, the second coating having at least one antibiotic agent dispersed in the second polymer such that the second polymer releases the at least one antibiotic agent as the second polymer degrades, wherein the second coating is configured to release the second active agent in over at least about 3 days; and
a third coating that covers at least a portion of the second coating, the third coating comprising a third biodegradable or bioresorbable polymer, the third coating having a hemostatic agent dispersed in the third polymer such that the third polymer releases the hemostatic agent as the third polymer degrades, wherein the third coating is configured to release the hemostatic agent in less than 3 days.
